(19) United States
(12) Patent Application Publication
(10) Pub. No.: US 2010/0048944 A1
(43) Pub. Date: Feb. 25, 2010

(54) INTERACTIONS OF HEDGEHOG AND LIVER X RECEPTOR SIGNALING PATHWAYS

(76) Inventor: Farhad Parhami, Los Angeles, CA (US)

Correspondence Address: VENABLE LLP, P.O. BOX 34385, WASHINGTON, DC, 20043-9998, US

(21) Appl. No.: 12/374,296

(22) PCT Filed: Jul. 19, 2007
(86) PCT No.: PCT/US07/16309

§ 371 Date: Jun. 30, 2009

Related U.S. Application Data
(60) Provisional application No. 60/832,290, filed on Jul. 19, 2006.

(51) Int. Cl. C07C 229/00; G01N 33/53; C12Q 1/34

(52) U.S. Cl. 562/441; 435/7.1; 435/18

(57) ABSTRACT

This invention relates to methods for using agents that are hedgehog inhibiting liver X receptor (LXR) agonists to reduce hedgehog signaling effects, such as cell proliferation, and methods for using the compounds, including treating subjects in need thereof, and pharmaceutical compositions and kits for implementing methods of the invention.

This application claims the benefit of the filing date of U.S. provisional application 60/832,290, filed Jul. 19, 2006 which is incorporated by reference herein.

Aspects of the invention were made with U.S. government support provided by NIH/NIAMS grant number R01-AR050426. The government has certain rights in the invention.

### BACKGROUND

Hedgehog molecules have been shown to play key roles in a variety of physiological processes including tissue patterning, mitogenesis, morphogenesis, cellular differentiation, differentiation of stem cells into mature cells, embryonic developments, and bone formation. In addition to its role in embryonic development, hedgehog signaling plays a crucial role in postnatal development and maintenance of tissue/organ integrity and function. Studies using genetically engineered mice have demonstrated that hedgehog signaling is important during skeletogenesis as well as in the development of osteoblasts in vitro and in vivo. In addition to playing a pro-osteogenic role, hedgehog signaling also inhibits adipogenesis when applied to pluripotent mesenchymal cells, C3H-10T ½.

Hedgehog signaling involves a very complex network of signaling molecules that includes plasma membrane proteins, kinases, phosphatases, and factors that facilitate the shuffling and distribution of hedgehog molecules. Production of hedgehog molecules from a subset of producing/signaling cells involves its synthesis, autoprocessing, and lipid modification. Lipid modification of hedgehog, which appears to be essential for its functionality, involves the addition of a cholesterol molecule to the C-terminal domain of the auto-cleaved hedgehog molecule and palmitoylation at its N-terminal domain. Additional accessory factors help shuttle hedgehog molecules to the plasma membrane of the signaling cells, release them into the extracellular environment, and transport them to the responding cells.

In the absence of hedgehog molecules, Patched (Ptch), present on the plasma membrane of the responding cells, keeps hedgehog signaling in a silent mode by inhibiting the activity of another plasma membrane associated signal transducer molecule, Smoothened (Smo). In the presence of hedgehog, the inhibition of Smo by Ptch is alleviated and Smo transduces the signal for the regulation of transcription of hedgehog-regulated genes. This transcriptional regulation in part involves the Ci/Gli transcription factors that enter the nucleus from the cytoplasm after a very intricate interaction between the members of a complex of accessory molecules that regulate Gli and its conversion from a 75 kd transcriptional repressor to a 155 kd transcriptional activator. The details of this highly complex signaling network have been extensively reviewed. (Cohen (2003) Am J Med Gen 123A, 5-28; Mullor et al. (2002) Trends Cell Bio 12, 562-569).

### SUMMARY

A hedgehog-inhibiting agonist of liver X receptor may be contacted with a cell in an amount effective to inhibit hedgehog signaling in the cell, and the inhibition of hedgehog signaling is measured. For example, oxysterol induction of alkaline phosphatase production, osteocalcin mRNA expression, or hedgehog agonist induced expression of a gene selected from the group consisting of Gli-1 and Ptch in the cell can be inhibited relative to a baseline value. For example, the agonist at a concentration of 5 μM can reduce the expression of Gli-1 in an M2-10B4 cell stimulated with 100 ng/mol of recombinant sonic hedgehog by at least 50%.

For example, a hedgehog-inhibiting agonist of liver X receptor can be TO901317, GW3965, 22R-hydroxycholesterol, or another other oxysterol or oxysterol-based molecules that activates liver X receptor.

TO901317 can be present in the extracellular environment of the cell with which it is contacted at a concentration of at least about 1 μM, 5 μM, or 10 μM.

A method for identifying an LXR agonist that inhibits a hedgehog pathway-mediated activity includes screening a candidate LXR agonist for the ability to inhibit the activity of Gli1 promoter or alkaline phosphatase activity in an in vitro assay.

A method for inhibiting a hedgehog (Hh) pathway mediated response in a cell or tissue includes contacting the cell or tissue with an effective amount of a hedgehog inhibiting LXR agonist. The cell or tissue can be in vitro or in a subject.

A method for reducing proliferation of a cell includes contacting the cell with a hedgehog-inhibiting liver X receptor agonist such as GW3965, 22R-hydroxycholesterol, or another oxysterol or oxysterol-based molecule that activates liver X receptor in an amount effective to reduce the proliferation of the cell. For example, the hedgehog-inhibiting liver X receptor agonist can be GW3965.

A method for reducing proliferation of a cell can include the following: determining inhibition of hedgehog signaling by at least one liver X receptor agonist; selecting a liver X receptor agonist that inhibits hedgehog signaling; and contacting the hedgehog-inhibiting liver X receptor agonist with a cell in an amount effective to reduce the proliferation of the cell. The cell can be, for example, a benign tumor cell or a cancer cell. For example, the cell can be a basal cell carcinoma cell, medulloblastoma cell, small cell lung cancer cell, pancreatic cancer cell, stomach cancer cell, esophageal cancer cell, colorectal cancer cell, melanoma cell, bladder cancer cell, bone cancer cell, osteosarcoma cell, or a tissue thereof.

A method of treating a basal cell carcinoma in a subject can include administering to the subject an effective amount of a hedgehog-inhibiting liver X receptor agonist. The agonist can be administered topically.

A method for treating a subject in need of reducing cell proliferation, can include administering to the subject an effective amount of a pharmaceutical composition comprising a hedgehog inhibiting LXR agonist.

A kit can include a hedgehog-inhibiting liver X receptor agonist selected from the group consisting of TO901317, GW3965, 22R-hydroxycholesterol, or another oxysterol or oxysterol-based molecule or other agent that activates liver X receptor. The kit can include a label indicating use in treating cancer in an animal or human. The agonist can be in a pharmaceutical composition. The pharmaceutical composition can be in a container.

A method includes inducing liver X receptor overexpression in a tumor cell in an amount effective to reduce the cell division rate of the tumor cell. For example, the liver X receptor overexpression can be induced by virally infecting the tumor cells with an LXR overexpression plasmid. In addition, a hedgehog-inhibiting liver X receptor agonist can be administered to the tumor cell.

A method includes manipulating stem cells to overexpress a hedgehog-inhibiting liver X receptor agonist. The manipulated stem cells can be administered to a subject in need of treatment of a cancerous or tumorous disease state in an amount sufficient to treat the cancerous or tumorous disease state.

### BRIEF DESCRIPTION OF THE FIGURES

FIG. 1 presents a bar graph illustrating the effect of LXR ligand on activation of Gli-reporter activity.

FIG. 2A presents the effect of LXR activation by TO901317 on Shh- and SS-induced expression of the hedgehog target gene Gli-1.

FIG. 2B presents the effect of LXR activation by TO901317 on Shh- and SS-induced expression of the hedgehog target gene Ptch.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the spirit and scope of the invention. All references cited herein are incorporated by reference as if each had been individually incorporated.

The importance of hedgehog signaling in developmental processes, makes its modulation of interest in controlling these processes, for example, in the treatment of cancer.

Liver X receptors (LXRs) are members of the family of nuclear hormone receptors. They are involved in a variety of physiologic processes including lipid and glucose metabolism, cholesterol homeostasis, and inflammatory signaling. Two isoforms of LXR have been identified and are referred to as LXRα and LXRβ. In this text, a liver X receptor (LXR) agonist is a compound that stimulates LXRα, LXRβ, or both. More generally, the term “liver X receptor (LXR)” indicates LXRα, LXRβ, or both. Liver X receptors can be activated by certain oxysterols and pharmacological agents such as TO901317 and GW3965.

An LXR or hedgehog agonist may be a substance that binds to a receptor and triggers a response in a particular type of cell. A hedgehog inhibitor is a chemical or biological substance that can reduce or eliminate specific biological or biochemical processes, and “inhibiting” refers to the effect of such substances on such processes in a cell.

Activation of the hedgehog signaling pathway by specific oxysterol molecules can result in osteoblastic differentiation of pluripotent mesenchymal stem cells while inhibiting their differentiation into adipocytes. Dysregulated (aberrant) hedgehog signaling can cause several cancers and may play a role in the metastasis of tumors including basal cell carcinoma, melanoma, stomach cancer, bladder cancer, prostate cancer, and bone cancer, such as osteosarcoma. Aberrant hedgehog signaling can promote cell division and proliferation of cancerous and tumorous cells. Therefore, the control of hedgehog signaling offers a route for treating certain cancers and bone disorders. However, aside from small molecule antagonists of the hedgehog pathway that are under development (mostly by Curis), no other known therapeutics or strategies exist for achieving hedgehog signaling inhibition.

Treatment of bone marrow stromal cells (MSC) with TO901317 or GW3965 can inhibit spontaneous osteogenic differentiation of these cells. LXR activation can inhibit osteoblast differentiation and bone formation. LXR activation can influence osteoclast differentiation and bone formation; LXR^{-/-} mice demonstrate an improved cortical bone phenotype.

The experiments discussed herein indicate that the activation of the nuclear hormone receptor, liver X receptor (LXR), by certain pharmacological or endogenous ligands can inhibit hedgehog signaling in a controlled manner. Activation of LXR therefore offers a route to interfering with dysregulated hedgehog signaling for the treatment of disease. For example, inhibition of the hedgehog pathway through activation of LXR is a mechanism for inhibiting hedgehog signaling which can be used to treat diseases and disorders such as certain cancers and skeletal diseases or defects.

Specific and/or non-specific activators of LXR family activity in a variety of cells can be used to modulate hedgehog signaling in those cells. For example, in the case of basal cell carcinoma, a topical application of LXR activators could inhibit the increased hedgehog pathway activity that appears to be a cause of the disease.

Certain osteogenic oxysterols, such as 20(S)-hydroxycholesterol (20S), induce the osteogenic differentiation of marrow stromal cells by activating the hedgehog signaling pathway. TO901317 and GW3965 block oxysterol-induced osteogenic differentiation of marrow stromal cells. Thus, LXR activation can interfere with osteogenic differentiation induced by the activation of hedgehog signaling.

N-(2,2,2-trifluoroethyl)-N-[4-(2,2,2-trifluoro-1-hydroxy-1-trifluoromethylethyl)phenyl]sulfonamide, also known as TO901317 (Calbiochem of EMD Biosciences, Inc.), is a cell-permeable, nonsterol, benzenesulfonamide compound that is a liver X receptor (LXR) agonist. TO901317 is a potent agonist, with a reported EC₅₀ of 20 nM for LXRα. 3-[3-[[[2-chloro-3-(trifluoromethyl)phenyl]methyl](2,2-diphenylethyl)amino]propoxy]benzeneacetic acid, also known as GW3965 (Glaxo SmithKline), is a synthetic dual LXRα_β agonist. Although TO901317 and GW3965 have been investigated as agents to control and treat inflammatory conditions, such as artherosclerosis, and metabolic disorders, such as diabetes, they have not been previously considered for a role in modulating hedgehog pathway activation.

The experiments discussed below indicate that TO901317 acts as an LXR agonist that has the net effect of inhibiting the hedgehog pathway. Thus, TO901317 is a hedgehog-inhibiting LXR agonist. GW3965 also acts as an LXR agonist, and has the net effect of inhibiting the hedgehog pathway. Thus, GW3965 is a hedgehog-inhibiting LXR agonist. 22(R)-hydroxycholesterol is an LXR agonist, and has the net effect of inhibiting the hedgehog pathway. Thus, 22(R)-hydroxycholesterol is a hedgehog-inhibiting LXR agonist. However, certain compounds that act as LXR agonists have the net effect of leaving the hedgehog pathway active or activating the hedgehog pathway. For example, 25-hydroxycholesterol is an LXR agonist, but leaves the hedgehog pathway active. 20(S)-hydroxycholesterol and the hydroxycholesterol Oxy13 (of which the structure is shown below) are LXR agonists, but have the net effect of activating the hedgehog pathway. The Oxy13 compound is discussed in international application PCT/US2007/05073, which is hereby incorporated by reference.

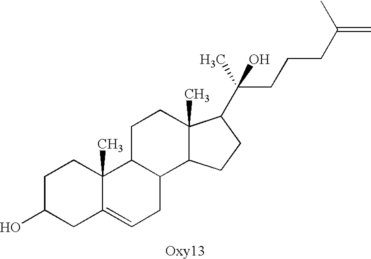

For the treatment of conditions, diseases, or disorders in which aberrant hedgehog signaling is implicated and hedgehog activation should be controlled or inhibited, in general the use of compounds that activate the LXR pathway and have the net effect of inhibiting the hedgehog pathway, that is, hedgehog-inhibiting LXR agonists, is indicated.

The experiments discussed demonstrate the effect of activation of LXR on associated physiological processes and reporter activity to assess modulation of the hedgehog pathway.

The present invention relates, e.g., to methods for using known and novel agents that are hedgehog inhibiting LXR agonists by contacting cells with such agents, to reduce cell proliferation and to treat other conditions mediated by elements of the hedgehog pathway.

One embodiment is a pharmaceutically acceptable composition that comprises at least such an agent, optionally in combination with other hedgehog inhibiting LXR agonists and/or other active agents.

As used herein, the singular forms “a,” “an” and “the” include plural referents unless the context clearly dictates otherwise. For example, “an” agonist includes multiple molecules, e.g. 2, 3, 4, 5 or more agonists.

Another aspect of the invention is a method for inhibiting a hedgehog (Hh) pathway mediated response in a cell or tissue, comprising contacting the cell or tissue with an effective amount of an agent or a pharmaceutical composition according to the invention. The cell or tissue may be in vitro or in a subject (in vivo). In the latter case, the subject can be one who would benefit from inhibition of hedgehog signaling, e.g. the inhibition of cell proliferation.

A “subject,” as used herein, includes any animal that exhibits a symptom of a condition that can be treated with a hedgehog inhibiting LXR agonist of the invention. Suitable subjects (patients) include laboratory animals (such as mouse, rat, rabbit, or guinea pig), farm animals, and domestic animals or pets (such as a cat or dog). Non-human primates and, preferably, human patients, are included. Typical subjects include animals that exhibit aberrant amounts (higher amounts than a “normal” or “healthy” subject) of one or more physiological activities that are stimulated by hedgehog signaling. The aberrant activities may be regulated by any of a variety of mechanisms, including activation of a hedgehog activity. The aberrant activities can result in a pathological condition.

An “effective amount,” as used herein, includes an amount that can bring about a detectable effect. A “therapeutically effective amount,” as used herein, includes an amount that can bring about a detectable therapeutic effect (e.g. the amelioration of a symptom).

Another aspect of the invention is a method for treating a subject suffering from a condition known to be mediated by the hedgehog pathway, comprising administering to the subject an effective amount of oxysterol or a pharmaceutical composition of the invention. Some such conditions are discussed elsewhere herein.

Another aspect of the invention is a method for identifying a hedgehog inhibiting LXR agonist, comprising screening candidate LXR agonist compounds for the ability to inhibit hedgehog activity in one of the hedgehog-related in vitro assays such as induction of expression of the Gli-1 gene, for example by stimulation of a Gli1 promoter; activation of a reporter construct driven by a multimerized Gli-1 responsive element; induction of expression of Patched; inhibition of a putative oxysterol-induced effect by cyclopamine, and other known methods.

Another aspect of the invention is in a method for inhibiting hedgehog (Hh) pathway mediated response in a cell or tissue (in vitro or in a subject), the improvement comprising contacting the cell or tissue with a hedgehog inhibiting LXR agonist of the invention. Another aspect of the invention is a method for treating a subject for one of the indications as described herein for example to reduce proliferation of cells, for example cancer cells.

A variety of conditions can be treated by compounds of the invention. Without being bound by any particular mechanism, it is suggested that among the conditions that can be treated by methods of the invention are cancers whose growth and/or metastasis can be inhibited by inhibition of hedgehog signaling, including, e.g., basal cell carcinoma (e.g., using a topical formulation) or other solid tumors, including medulloblastoma, small cell lung cancer, pancreatic cancer, stomach cancer, esophageal cancer, colorectal cancer, prostate cancer and breast cancer (e.g., using a systemic formulation).

The agents discussed herein can be formulated into various compositions, e.g., pharmaceutical compositions, for use in therapeutic treatment methods. The pharmaceutical compositions can be assembled as a kit. Generally, a pharmaceutical composition of the invention comprises an effective amount of a hedgehog inhibiting LXR agonist or combination of the invention. An “effective amount,” as used herein, is an amount that is sufficient to effect at least a detectable therapeutic response in the individual over a reasonable time frame. For example, it can ameliorate, at least to a detectable degree, the symptoms of a hedgehog-mediated condition, etc. An effective amount can prevent, reduce, treat, or eliminate the particular condition.

The composition can comprise a carrier, such as a pharmaceutically acceptable carrier. By “pharmaceutically acceptable” is meant a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. For a discussion of pharmaceutically acceptable carriers and other components of pharmaceutical compositions, see, e.g., Remington's Pharmaceutical Sciences, 18th ed., Mack Publishing Company, 1990.

A pharmaceutical composition or kit of the invention can contain other pharmaceuticals, in addition to the hedgehog inhibiting agents of the invention. The other agent(s) can be administered at any suitable time during the treatment of the patient, either concurrently or sequentially.

One skilled in the art will appreciate that the particular formulation will depend, in part, upon the particular agent that is employed, and the chosen route of administration. Accordingly, there is a wide variety of suitable formulations of compositions of the present invention.

Formulations suitable for oral administration can consist of liquid solutions, such as an effective amount of the agent dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract.

Formulations suitable for parenteral administration (e.g. intravenous) include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The hedgehog inhibiting LXR agonists of the invention, alone or in combination with other therapeutic agents, can be made into aerosol formulations to be administered via inhalation. These aerosol formulations can be placed into pressurized acceptable propellants, such as dichlorodifluoromethane, propane, nitrogen and the like.

The hedgehog inhibiting LXR agonists of the invention, alone or in combinations with other therapeutic agents, can be made into suitable formulations for transdermal application and absorption (Wallace et al., 1993, supra). Transdermal electroporation or iontophoresis also can be used to promote and/or control the systemic delivery of the agents and/or pharmaceutical compositions of the present invention through the skin (e.g., see Theiss et al. (1991), Meth. Find. Exp. Clin. Pharmacol. 13, 353-359).

Formulations which are suitable for topical administration include lozenges comprising the active ingredient in a flavor, usually sucrose and acacia or tragacanth; pastilles comprising the active ingredient in an inert base, such as gelatin and glycerin, or sucrose and acacia; mouthwashes comprising the active ingredient in a suitable liquid carrier; or creams, emulsions, suspensions, solutions, gels, creams, pastes, foams, lubricants, sprays, suppositories, or the like.

One skilled in the art will appreciate that a suitable or appropriate formulation can be selected, adapted or developed based upon the particular application at hand.

Dosages for hedgehog inhibiting LXR agonists of the invention can be in unit dosage form, such as a tablet or capsule. The term “unit dosage form” as used herein refers to physically discrete units suitable as unitary dosages for animal (e.g. human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle.

One skilled in the art can easily determine the appropriate dose, schedule, and method of administration for the exact formulation of the composition being used, in order to achieve the desired effective amount or effective concentration of the agent in the individual patient. One skilled in the art also can readily determine and use an appropriate indicator of the “effective concentration” of the compounds of the present invention by a direct or indirect analysis of appropriate patient samples (e.g., blood and/or tissues). Assays of hedgehog inhibition can calibrate dosage for particular LXR agonists.

The dose of a hedgehog inhibiting LXR agonist of the invention, or composition thereof, administered to an animal, particularly a human, in the context of the present invention should be sufficient to effect at least a therapeutic response in the individual over a reasonable time frame. The dose used to achieve a desired concentration in vivo will be determined by the potency of the particular hedgehog inhibiting LXR agonist employed, the pharmacodynamics associated with the agent in the host, the severity of the disease state of infected individuals, as well as, in the case of systemic administration, the body weight and age of the individual. The size of the dose also will be determined by the existence of any adverse side effects that may accompany the particular agent, or composition thereof, employed. It is generally desirable, whenever possible, to keep adverse side effects to a minimum.

For example, a dose can be administered in the range of from about 5 ng (nanograms) to about 1000 mg (milligrams), or from about 100 ng to about 600 mg, or from about 1 mg to about 500 mg, or from about 20 mg to about 400 mg. For example, the dose can be selected to achieve a dose to body weight ratio of from about 0.0001 mg/kg to about 1500 mg/kg, or from about 1 mg/kg to about 1000 mg/kg, or from about 5 mg/kg to about 150 mg/kg, or from about 20 mg/kg to about 100 mg/kg. For example, a dosage unit can be in the range of from about 1 ng to about 5000 mg, or from about 5 ng to about 1000 mg, or from about or from about 100 ng to about 600 mg, or from about 1 mg to about 500 mg, or from about 20 mg to about 400 mg, or from about 40 mg to about 200 mg of a compound of according to the present invention. A dose can be administered once per day, twice per day, four times per day, or more than four times per day as required to elicit a desired therapeutic effect. For example, a dose administration regimen can be selected to achieve a blood serum concentration of a compound of the present invention in the range of from about 0.01 to about 20000 nM, or from about 0.1 to about 15000 nM, or from about 1 to about 10000 nM, or from about 20 to about 10000 nM, or from about 100 to about 10000 nM, or from about 200 to about 5000 nM, or from about 1000 to about 5000 nM. For example, a dose administration regime can be selected to achieve an average blood serum concentration with a half maximum dose of a compound of the present invention in the range of from about 1 μg/L (microgram per liter) to about 2000 μg/L, or from about 2 μg/L to about 1000 μg/L, or from about 5 μg/L to about 500 μg/L, or from about 10 μg/L to about 400 μg/L, or from about 20 μg/L to about 200 μg/L, or from about 40 μg/L to about 100 μg/L.

A therapeutically effective dose of a hedgehog inhibiting LXR agonist or other agent useful in this invention is one which has a positive clinical effect on a patient as measured by the ability of the agent to reduce cell proliferation. The therapeutically effective dose of each agent can be modulated to achieve the desired clinical effect, while minimizing negative side effects. The dosage of the agent may be selected for an individual patient depending upon the route of administration, severity of the disease, age and weight of the patient, other medications the patient is taking and other factors normally considered by an attending physician, when determining an individual regimen and dose level appropriate for a particular patient.

When given in combined therapy, the other agent can be given at the same time as the hedgehog inhibiting LXR agonist, or the dosing can be staggered as desired. The two (or more) drugs also can be combined in a composition. Doses of each can be less when used in combination than when either is used alone.

The invention may include treatment with an additional agent which acts independently or synergistically with the hedgehog inhibitor. Additional classes of agents which may be useful in this invention alone or in combination with hedgehog inhibiting LXR agonists include, but are not limited to known anti-proliferative agents. Those skilled in the art would be able to determine the accepted dosages for each of the therapies using standard therapeutic dosage parameters.

The invention may include a method of systemic delivery or localized treatment alone or in combination with administration of other agent(s) to the patient.

Another embodiment of the invention is a kit useful for any of the methods disclosed herein, either in vitro or in vivo. Such a kit can comprise one or more of the hedgehog inhibiting LXR agonists or pharmaceutical compositions discussed herein. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form. A skilled worker will recognize components of kits suitable for carrying out any of the methods of the invention.

In the foregoing and in the following examples, all temperatures are set forth in uncorrected degrees Celsius; and, unless otherwise indicated, all parts and percentages are by weight.

The hedgehog inhibiting LXR agonist may be a compound other than TO0901317, identified according to methods of the invention. The cells and tissues in which cell proliferation is inhibited may be basal cell carcinoma, medulloblastoma, small cell lung cancer, pancreatic cancer, stomach cancer, esophageal cancer, or colorectal cancer cells or tissues. It may be a cancer other than prostate cancer or breast cancer.

The inventive methods can be used to treat cell proliferative disorders mediated by hedgehog signaling. “Cell proliferative disorders” refer to disorders wherein unwanted cell proliferation of one or more subset(s) of cells in a multicellular organism occurs, resulting in harm (e.g., discomfort or decreased life expectancy) to the multicellular organism. Cell proliferative disorders can occur in different types of animals and in humans. Cell proliferative disorders include cancers in particular. A “therapeutic effect” generally refers to either the inhibition, to some extent, of growth of cells causing or contributing to a cell proliferative disorder; or the inhibition, to some extent, of the production of factors (e.g., growth factors) causing or contributing to a cell proliferative disorder. A therapeutic effect relieves to some extent one or more of the symptoms of a cell proliferative disorder. In reference to the treatment of a cancer, a therapeutic effect may refer to one or more of the following: 1) reduction in the number of cancer cells; 2) reduction in tumor size; 3) inhibition (i.e., slowing to some extent, preferably stopping) of cancer cell infiltration into peripheral organs; 3) inhibition (i.e., slowing to some extent, preferably stopping) of tumor metastasis; 4) inhibition, to some extent, of tumor growth; and/or 5) relieving to some extent one or more of the symptoms associated with the disorder.

### General Assay Approach

In general, the following methods and approaches can be used to determine whether a compound is a liver X receptor (LXR) agonist that inhibits the hedgehog pathway (i.e., a hedgehog-inhibiting liver X receptor agonist).

The compound can be tested for LXR agonistic character by various assay techniques. For example, the compound can be tested with an LXR specific reporter assay, for example, an assay that expresses luciferase upon activation of the LXR pathway. One of skill in the art would know how to conduct a specific reporter assay. If the compound causes the expression of luciferase to increase above that of a control cell by a statistically significant amount, then the compound is deemed an LXR agonist. As another example, the compound can be tested by measuring (for example, by quantitative real time PCR) the expression of LXR target genes in a model cell contacted with the compound. One of skill in the art would know how to measure the expression of genes in model cell, and how to conduct quantitative real time PCR. If the compound causes expression of the LXR target genes to increase above that of a control cell by a statistically significant amount, then the compound is deemed an LXR agonist. Examples of such LXR target genes are ABCA1 and ABCG1, which are ATP binding transporters. Any cell that responds to an LXR agonist can be used as a model cell. For example, marrow stromal cells can be used as model cells.

In order to identify a hedgehog-inhibiting LXR agonist, certain assays make use of a hedgehog agonist in the assay. The hedgehog agonist stimulates hedgehog signaling in a cell. Any hedgehog-inhibiting character of an LXR agonist is then noted by observing a decrease of hedgehog signaling in the hedgehog stimulated cell.

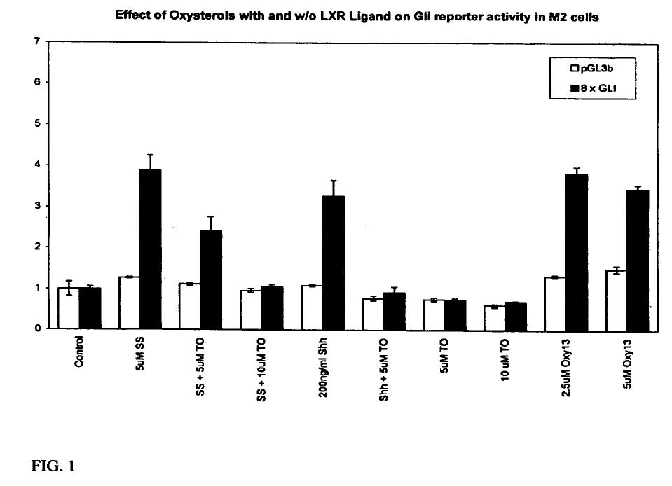

For example, to determine whether a compound that is an LXR agonist has the net effect of inhibiting the hedgehog pathway, the following method can be used. A known hedgehog agonist (hedgehog agonists that can be used in the method are known in the art) is contacted with a model cell in an amount known to induce a hedgehog pathway response in the cell. Any cell that responds to a hedgehog agonist and an LXR agonist can be used as a model cell, for example, marrow stromal cells can be used. The compound is then tested with a hedgehog specific reporter assay, for example, an assay that expresses luciferase upon activation of the hedgehog pathway. The hedgehog agonist will cause the expression of luciferase to increase above that of a control cell. While the hedgehog agonist is in contact with the cell, the compound being tested is also contacted with the cell. The expression of luciferase is measured. If introducing the compound decreases the model cell's expression of luciferase a statistically significant amount below the expression of the luciferase when only the hedgehog agonist was in contact with the cell, then the compound is deemed a hedgehog-inhibiting LXR agonist. An example of this method is described in the section below entitled “In Vitro Study of Hedgehog Inhibition by LXR Agonist Using Gli Reporter Assay,” and results shown in FIG. 1.

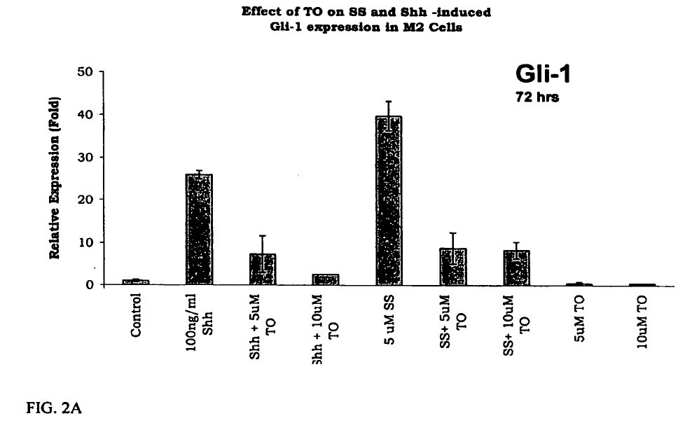

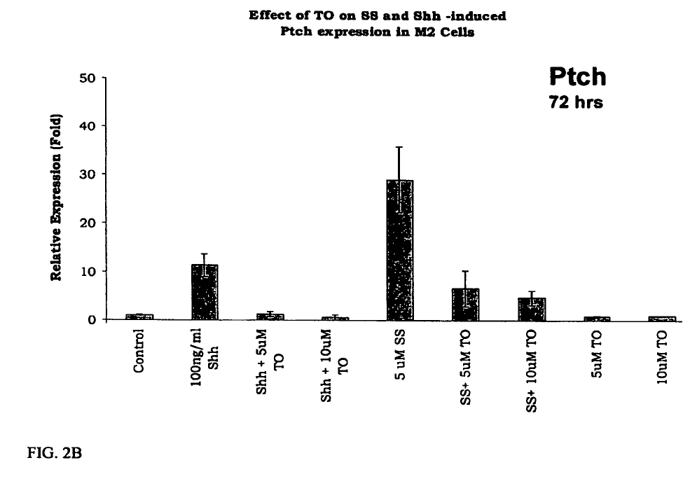

Another example of a method to determine whether a compound that is an LXR agonist has the net effect of inhibiting the hedgehog pathway is the following. A known hedgehog agonist is contacted with a model cell in an amount known to induce a hedgehog pathway response in the cell. Any cell that responds to a hedgehog agonist and an LXR agonist can be used as a model cell, for example, marrow stromal cells can be used. An assay is conducted to measure the expression of a hedgehog target gene, such as Gli-1 or Ptch, which will be increased above that of a control cell. One of skill in the art would know how to conduct an assay to measure the expression of a target gene. While the hedgehog agonist is in contact with the cell, the compound being tested is also contacted with the cell. The expression of the hedgehog target gene is measured. If introducing the compound decreases the model cell's expression of the hedgehog target gene a statistically significant amount below the expression of the gene when only the hedgehog agonist was in contact with the cell, then the compound is deemed a hedgehog-inhibiting LXR agonist. An example of this method is described in the section below entitled “In Vitro Study of Hedgehog Inhibition by LXR Agonist Using Measurement of Hedgehog Target Gene Expression,” and results shown in FIGS. 2A and 2B.

More generally, the following approach can be used to measure inhibition of hedgehog signaling and determine whether a compound that is an LXR agonist has the net effect of inhibiting the hedgehog pathway or inhibiting hedgehog signaling. A known hedgehog agonist is contacted with a model cell in an amount known to induce a hedgehog pathway response or hedgehog signaling response in the cell. Any cell that responds to a hedgehog agonist and an LXR agonist can be used as a model cell. The compound is then tested with an assay to measure the level of hedgehog activation or hedgehog signaling. One of skill in the art would know how to select an assay and conduct an assay to measure the level of hedgehog activation or hedgehog signaling. The hedgehog agonist will cause the level of hedgehog activation or signaling to increase above that of a control cell. While the hedgehog agonist is in contact with the cell, the compound being tested is also contacted with the cell. The level of hedgehog activation or signaling is measured. If introducing the compound decreases the level of hedgehog activation or signaling a statistically significant amount below the level when only the hedgehog agonist was in contact with the cell, then the compound is deemed a hedgehog-inhibiting LXR agonist.

What a “statistically significant amount” is depends on the a number of factors, such as the technique of the experimenter and the quality of the equipment used. For example, in certain cases, a statistically significant amount may be a change of 1%. In other cases, a statistically significant amount can be represented by a change of at least about 5%, 10%, 20%, 50%, 75%, double, or more. In relation to inhibition, the significant reduction may be to a level of less than about 90%, 75%, 50%, 25%, 10%, 5%, 1%, or less.

### EXAMPLES

### In Vitro Study of Effect of LXR Activation on Alkaline Phosphatase Activity

Alkaline phosphatase activity provides a measure of the osteogenic differentiation of cells. The treatment of pluripotent marrow stromal cells, M2-10B4, with recombinant sonic hedgehog (herein, Shh) at a concentration of 50 ng/ml induced the activity of alkaline phosphatase, as illustrated in Table 1. The oxysterol 20(S)-hydroxycholesterol (herein, 20S) was also found to induce alkaline phosphatase activity in the marrow stromal cells. A combination of 22(S)-hydroxycholesterol (herein, 22S) and 20(S)-hydroxycholesterol (the combination herein, SS) at a concentration of 5 μM was found to induce alkaline phosphatase activity in the marrow stromal cells, as shown in Table 1. The oxysterol combination SS contains 22(S)-hydroxycholesterol and 20(S)-hydroxycholesterol in equal molar proportions. An indication of the molar concentration of the oxysterol combination SS refers to the molar concentration of each component of the combination. For example, a 5 μM concentration of SS indicates that 5 μM of 22(S)-hydroxycholesterol and 5 μM of 20(S)-hydroxycholesterol are present in the solution.

TABLE 1

| | Alkaline Phosphatase Activity |
|---|---|
| Treatment | (units/mg protein ± SD) |
| Control | 0 |
| Shh (50 ng/ml) | 185 ± 3 |
| Shh (50 ng/ml) + TO901317 (5 μM) | 3 ± 2 |
| Shh (50 ng/ml) + TO901317 (10 μM) | 0 |
| SS (5 μM) | 726 ± 43 |
| SS (5 μM) + TO901317 (5 μM) | 198 ± 17 |
| SS (5 μM) + TO901317 (10 μM) | 61 ± 12 |
| TO901317 (5 μM) | 0 |
| TO901317 (10 μM) | 0 |

Treatment of the M2-10B4 marrow stromal cells with TO901317 inhibited alkaline phosphatase activation by Shh and oxysterol, as shown in Table 1. That is, whereas dosing the cells with 50 ng/mL of Shh induced an alkaline phosphatase activity of 185±3 units/mg protein, dosing the cells with 5 μM of TO901317 with the same concentration of Shh lowered the alkaline phosphatase activity to 3±2 units/mg protein. The alkaline phosphatase activity when stimulated by a hedgehog agonist such as Shh represents a baseline value. The observation of the decrease in alkaline phosphatase activity upon addition of the LXR agonist TO901317 represents inhibition of alkaline phosphatase production in the cell relative to the baseline value.

Furthermore, dosing the cells with 10 μM of TO901317 with the same concentration of Shh lowered the alkaline phosphatase activity to where it could not be detected. Similarly, whereas dosing the M2-10B4 marrow stromal cells with 5 μM of SS induced an alkaline phosphatase activity of 726±43 units/mg protein, dosing the cells with 5 μM of the oxysterol combination SS with the same concentration of Shh lowered the alkaline phosphatase activity to 198±17 units/mg protein. Increasing the concentration of SS to 10 μM resulted in a further reduction of alkaline phosphatase activity to 61±12 units/mg protein.

Treatment of M2-10B4 marrow stromal cells for 3 days with TO901317 in concentrations as low as 1 μM inhibited sonic hedgehog (Shh)-induced alkaline phosphatase activity. Shh-induced osteocalcin mRNA expression was inhibited relative to a baseline value in which the cells were treated with Shh alone by treatment of M2-10B4 cells with TO901317 for 3 days in concentrations ranging from 1 μM to 10 μM. Similar results were obtained using a second multipotent cell line, the C3H10T½ mouse embryonic fibroblasts in which TO901317 (1-10 μM) inhibited alkaline phosphatase activity and osteocalcin expression.

The control cells to which neither sonic hedgehog (Shh), the oxysterol combination SS, nor the hedgehog-inhibiting LXR agonist TO901317 were added exhibited no detectable alkaline phosphatase activity. Cells to which only TO901317, in concentrations of 5 μM or 10 μM, was added exhibited no detectable alkaline phosphatase activity, demonstrating that TO901317 does not stimulate hedgehog activity.

The hedgehog-inhibiting liver X receptor agonist GW3965 also inhibited sonic hedgehog (Shh)-induced osteogenic differentiation of M2-10B4 marrow stromal cells. When treated with from 1-10 μM of GW3965 for 8 days, GW3965 inhibited Shh-induced alkaline phosphatase activity.

Because both Shh and oxysterols such as 20S and 22S act to stimulate alkaline phosphatase activity through activation of the hedgehog pathway, and the hedgehog-inhibiting liver X receptor agonists TO901317 and GW3965 acted to decrease this stimulated alkaline phosphatase activity, these results demonstrated that hedgehog pathway activation is inhibited by the activation of the liver X receptor.

### In Vitro Study of Hedgehog Inhibition by LXR Agonist Using Gli Reporter Assay

Activation or inhibition of the hedgehog pathway was assessed by measuring Gli-reporter activity. This provided a direct indication of the activation or inhibition of the hedgehog pathway.

A first set of marrow stromal cells, M2-10B4, were transfected with a Gli-luciferase reporter construct (8Xgli) and a second set of M2-10B4 were transfected with an empty reporter construct without Gli response elements (pGL3b). The first and second sets of cells were then treated with sonic hedgehog (Shh) or the oxysterol combination SS and were treated either with no LXR agonist, with an intermediate concentration of the hedgehog-inhibiting LXR agonist TO901317, or with a high concentration of the hedgehog-inhibiting LXR agonist TO901317. Gli-luciferase reporter activity was tested after 48 hours of treatment.

FIG. 1 presents the results of the study. Results from a representative experiment are shown as the mean of triplicate determinations±SD, and expressed as fold induction over control untreated cells. Treatment with 200 ng/mL of Shh resulted in an increase in Gli-luciferase reporter activity of about 3.5 times over that of the control cells; this represented a baseline value. Addition of 5 μM of TO901317 to the Shh treated cells reduced the Gli-luciferase reporter activity below the baseline value, and to about the same level as of the control cells.

Treatment with 5 μM of SS resulted in an increase in Gli-luciferase reporter activity of about 4 times over that of the control cells. Addition of 5 μM of TO901317 to the SS treated cells reduced the Gli-luciferase reporter activity to only about 2.5 times over that of the control cells. And addition of 10 μM of TO901317 to the SS treated cells further reduced the Gli-luciferase reporter activity to about the same level as of the control cells.

Addition of 5 μM or 10 μM of TO901317 to M2-10B4 cells resulted in the cells having Gli-luciferase reporter activity of less than the control cells, demonstrating that TO901317 does not stimulate hedgehog activity.

Because the hedgehog-inhibiting liver X receptor agonist TO901317 acted to decrease the Gli-luciferase reporter activity stimulated by sonic hedgehog (Shh) or the oxysterol combination (SS), these results directly demonstrated that hedgehog pathway activation is inhibited by the activation of the liver X receptor.

### In Vitro Study of Hedgehog Inhibition by LXR Agonist Using Measurement of Hedgehog Target Gene Expression

Activation or inhibition of the hedgehog pathway was assessed by extracting RNA from test cells and using quantitative real time PCR (Q-RT-PCR) to measure the expression of the hedgehog target genes, Gli-1 and Patched (Ptch).

Marrow stromal cells, M2-10B4, were treated with sonic hedgehog (Shh) or the oxysterol combination SS and were treated either with no LXR agonist, with an intermediate concentration of the hedgehog-inhibiting LXR agonist TO901317, or with a high concentration of TO901317. Expression of Gli-1 and Patched (Ptch) was measured after 72 hours of treatment.

FIGS. 2A and 2B present the results of the study. Results from a representative experiment are shown as the mean of triplicate determinations±SD, and expressed as fold induction over control untreated cells.

FIG. 2A presents the expression of Gli-1. Treatment with 100 ng/mL of recombinant sonic hedgehog (Shh) resulted in an increase in Gli-1 expression of about 25 times over that of the control cells, representing a baseline value. Addition of 5 μM of TO901317 to the Shh treated cells reduced the Gli-1 expression below the baseline value, to about 7 times over that of the control cells. Addition of 10 μM of TO901317 to the Shh treated cells further reduced the Gli-1 expression to about 3 times over that of the control cells. Treatment with 5 μM of the oxysterol combination SS resulted in an increase in Gli-1 expression of about 40 times over that of the control cells. Addition of 5 μM of TO901317 to the SS treated cells reduced the Gli-1 expression to about 8 times over that of the control cells. Addition of 10 μM of TO901317 to the SS treated cells reduced the Gli-1 expression to about 7 times over that of the control cells.

Addition of 5 μM or 10 μM of TO901317 to M2-10B4 cells resulted in the cells having Gli-1 expression of less than the control cells, demonstrating that TO901317 does not stimulate hedgehog activity.

FIG. 2B presents the expression of Ptch. Treatment with 100 ng/mL of recombinant sonic hedgehog (Shh) resulted in an increase in Ptch expression of about 12 times over that of the control cells, representing a baseline value. Addition of 5 μM of TO901317 to the Shh treated cells reduced the Ptch expression below the baseline value, to about the same level as that of the control cells. Addition of 10 μM of TO901317 to the Shh treated cells reduced the Ptch expression to about the same level as that of the control cells. Treatment with 5 μM of the oxysterol combination SS resulted in an increase in Ptch expression of about 30 times over that of the control cells. Addition of 5 μM of TO901317 to the SS treated cells reduced the Ptch expression to about 6 times over that of the control cells. Addition of 10 μM of TO901317 to the SS treated cells reduced the Gli-1 expression to about 4 times over that of the control cells.

Addition of 5 μM or 10 μM of TO901317 to M2-10B4 cells resulted in the cells having Ptch expression of about the same as the control cells, demonstrating that TO901317 does not stimulate hedgehog activity.

Because the hedgehog-inhibiting liver X receptor agonist TO901317 acted to decrease the expression of both Gli-1 and Ptch in cells stimulated by sonic hedgehog (Shh) or the oxysterol combination (SS), the results demonstrated that hedgehog pathway activation is inhibited by the activation of the liver X receptor.

### Study of Effect of siRNA on Liver X Receptor

Small interfering RNA (siRNA) to LXRα and LXRβ caused an 80-90% inhibition of mRNA expression for these genes, as well as inhibition of ligand-induced expression of LXR target genes, ATP-binding cassette (ABC) transporter proteins ABCA1 and ABCG1. The ability of TO901317 and GW3965 to inhibit Shh-induced signaling and alkaline phosphatase activity was blocked in cells transfected with LXRα and LXRβ siRNA, but not in cells transfected with control scrambled siRNA.

The experiments performed demonstrated that LXR activation can inhibit hedgehog signaling and osteogenic differentiation of marrow stromal cells. Liver X receptor appears to have a negative role in bone metabolism. Thus, modulation, for example, inhibition of hedgehog signaling by activating LXR can be a therapeutic strategy to treat malignancy/tumors of the skeletal system/bone that may be caused by aberrant hedgehog signaling and/or other mechanisms that result in uncontrolled growth/function of bone cells.

### Treatment by Overexpression of Liver X Receptor

The above experiments indicate that activation of liver X receptor (LXR) can inhibit the hedgehog pathway. As discussed above, aberrant expression of hedgehog is implicated in disease processes, such as division and proliferation of certain cancer or tumor cells. Therefore, the expression of liver X receptor in cancer or tumor cells in a subject can be induced as a therapy. For example, a therapy can stimulate overexpression of LXR in cancer or tumor cells, so that the liver X pathway tends to be overstimulated, and the hedgehog pathway inhibited, so that aberrant hedgehog pathway activity in stimulating division and proliferation of cancer or tumor cells is reduced. Cancer or tumor cells in which LXR is overexpressed are also likely to be more sensitive to liver X receptor agonists. Thus, a combination therapy could include treatment to induce overexpression of LXR in cancer or tumor cells, along with administration of an LXR agonist.

Overexpression of LXR in cancer or tumor cells can be induced by, for example, a gene therapy approach. For example, viral infection of cancer or tumor cells with an LXR overexpression plasmid to induce overexpression of LXR.

### Treatment by Targeted Delivery of Liver X Receptor Agonist

In order to minimize potential side effects, and maximize the concentration of liver X receptor agonist to which cancer or tumor cells are exposed, a method of treatment may use a targeted approach to deliver hedgehog-inhibiting LXR agonist directly to the cancer or tumor cells. For example, mechanical means can be used to deliver the hedgehog-inhibiting LXR agonist to the cancer cells. For example, a catheter can be inserted into or next to a tumor or region of cancerous cells, and the hedgehog-inhibiting LXR agonist administered at a controlled rate. A controlled release device can be implanted into or next to a tumor or region of cancerous cells, so that the hedgehog-inhibiting LXR agonist is released at a controlled rate. Alternatively, a biomolecular targeting approach can be used to deliver hedgehog-inhibiting LXR agonist to tumor or cancer cells. For example, stem cells tend to concentrate near proliferating cancer or tumor cells. Stem cells can be manipulated to express a hedgehog-inhibiting liver X receptor agonist at a high rate. Then, by administering the manipulated stem cells to a subject in need of treatment, the stem cells can concentrate around proliferating cancer or tumor cells, where they will release hedgehog-inhibiting liver X receptor agonist, so that the cancer or tumor cells are in contact with a high concentration of hedgehog-inhibiting liver X receptor agonist, inhibiting the hedgehog pathway and inhibiting proliferation of the cancer or tumor cells.

### Administration of Liver X Receptor Agonists

Hedgehog-inhibiting liver X receptor (LXR) agonists can be administered by any one of or a combination of several routes. For example, TO901317 and GW3965 can be administered orally, injected, e.g., injected intravascularly, or administered topically. For research purposes, the route of administration selected by the researcher can depend on the topic of study. For therapeutic purposes, the route of administration to a subject selected by the clinician can depend on, for example, the disease state, the extent of the disease, the general physical condition of the subject, and a number of other factors. For example, a hedgehog-inhibiting LXR agonist can be administered topically to the site of a basal cell carcinoma to treat this disease.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above are hereby incorporated by reference in their entirety.

1.-13. (canceled)

14. A method for identifying an LXR agonist that inhibits a hedgehog pathway-mediated activity, comprising screening a candidate LXR agonist for the ability to inhibit the activity of Gli1 promoter or alkaline phosphatase activity in an in vitro assay.

15. A method for inhibiting a hedgehog (Hh) pathway mediated response in a cell or tissue, comprising:
contacting the cell or tissue with an effective amount of a hedgehog inhibiting LXR agonist; and
measuring hedgehog inhibition.

16. The method of claim 15, wherein the cell or tissue is in vitro.

17. The method of claim 15, wherein the cell or tissue is in a subject.

18.-28. (canceled)

29. A method for identifying an LXR agonist that inhibits a hedgehog pathway-mediated activity, comprising screening a candidate LXR agonist for the ability to inhibit a hedgehog mediated activity in an in vitro assay and selecting a hedgehog inhibiting LXR agonist with a predetermined level of hedgehog inhibition.

30. A hedgehog-inhibiting liver X receptor agonist selected by the method of claim 29.

31. A hedgehog-inhibiting liver X receptor agonist of claim 30 that is not TO901317.

32.-40. (canceled)
